(19) United States
(12) Patent Application Publication
(10) Pub. No.: US 2013/0102855 A1
(43) Pub. Date: Apr. 25, 2013

(54) BODE INDEX MEASUREMENT

(71) Applicants: Amy Oi Mee Cheung, Eindhoven (NL); Maryam Atakhorrami, Cambridge (GB)

(72) Inventors: Amy Oi Mee Cheung, Eindhoven (NL); Maryam Atakhorrami, Cambridge (GB)

(73) Assignee: KONINKLIJKE PHILIPS ELECTRONICS N.V., EINDHOVEN (NL)

(21) Appl. No.: 13/513,007

(22) PCT Filed: Nov. 17, 2010
(86) PCT No.: PCT/IB10/55222

§ 371 Date: May 31, 2012

Related U.S. Application Data
(60) Provisional application No. 61/288,370, filed on Dec. 21, 2009.

(51) Int. Cl. A61B 5/08; A61B 5/11; A61B 5/00

(52) U.S. Cl. CPC A61B 5/0826; A61B 5/4872; A61B 5/0816; A61B 5/1118; A61B 5/7225; A61B 5/6831; A61B 5/6804
USPC 600/301

(57) ABSTRACT

A computer-implemented system and method for determining a BODE index value for a patient is provided. The method includes using a Body Mass Index (BMI) measuring device to obtain BMI data. An airway obstruction measuring device measures airway obstruction of the patient to obtain airway obstruction data. A respiration rate sensor measures a respiration rate of the patient and to obtain respiration rate data. An activity monitor measures physical activity of the patient and to obtain physical activity data. A processor executes a computer program module to determine the BODE index value for the patient based on the Body Mass Index data, the airway obstruction data, the respiration rate data, and the physical activity data.

This patent application claims the priority benefit under 35 U.S.C. §119(e) of U.S. Provisional Application No. 61/288,370 filed on Dec. 21, 2009, the contents of which are herein incorporated by reference.

The present invention relates to a method and a system for determining a BODE index value for a patient.

Chronic Obstructive Pulmonary Disease (COPD) is a respiratory disease that is characterized by inflammation of the airways. It is characterized by an airflow limitation that is not fully reversible. The airflow limitation is both progressive and associated with an abnormal inflammatory response of the lungs to noxious particles or gases. Symptoms of COPD may include coughing, wheezing and the production of mucus and the degree of severity may, in part, be viewed in terms of the volume and color of secretions.

Exacerbations are the worsening of COPD symptoms. The exacerbations may be associated with a variable degree of physiological deterioration. The exacerbations may be characterized by increased coughing, dyspnea (i.e., shortness of breath) and production of sputum. Typically, exacerbations are confirmed by a general practitioner or a hospital physician, or detected via questionnaires.

A BODE index is a four-dimensional grading system for COPD severity. The BODE index is based on four parameters: 1) Body Mass Index (B), 2) degree of obstruction (O), 3) dyspnea (D), and 4) exercise capacity (E). The BODE index provides valuable prognostic information after pulmonary rehabilitation of a COPD patient. Studies have indicated that COPD patients have an improved BODE index after completion of the pulmonary rehabilitation. Furthermore, the BODE index is also a good predictor of mortality, frequency and severity of exacerbations.

The Body Mass Index is generally obtained by dividing the patient's body weight by the square of patient's height. The degree of obstruction in the patient's airways is measured by a Forced Expiratory Volume measured over one second (FEV₁) value (using a spirometry test). The level of dyspnea (i.e., shortness of breath) in the patient is determined using a dyspnea questionnaire. A six minute walk test is used to evaluate the exercise capacity of the patient.

A point value is assigned to each parameter (i.e., Body Mass Index (B), degree of obstruction (O), dyspnea (D), and exercise capacity (E)) and the point values are added to obtain the BODE index. The BODE index generally ranges from 0-10 points, with higher scores indicating a greater risk of death, see TABLE. 1 below.

TABLE 1

| BODE | Points | | | | |
|---|---|---|---|---|---|
| parameters | 1 | 2 | 3 | 4 | Score |
| Body Mass | <21 | ≧21 | | | |
| Index, BMI | | | | | |
| Airway | >65% | 54-64% | 36-49% | ≦35% | |
| obstruction | | | | | |
| (measured in | | | | | |
| FEV₁) | | | | | |
| Dyspnea | 0-1 | 2 | 3 | 4 | |
| Questionnaire | | | | | |
| Six minute walk | ≧350 m | 250-349 m | 150-249 m | ≦149 m | |
| test (distance | | | | | |
| measured in | | | | | |
| meters) | | | | | |
| | | | | Total | |

The measurement of the BODE index requires a clinician to administer the dyspnea questionnaire and to evaluate the six minute walk test (e.g., using two cones (that are separated by a distance of thirty meters), and a stopwatch). As a result, in order to calculate their BODE index, the patient would typically attend a clinic or a pulmonary rehabilitation center, and consequently, the BODE index is typically not acquired on a frequent basis. In addition, questionnaire based assessments, such as one used for dyspnea, are subjective and rely on memory recall, which is especially more difficult for the elderly.

Accordingly, it is an object of the present invention to provide a system and method of determining a BODE index value that overcomes the shortcomings of conventional techniques. This object is achieved according to one embodiment of the present invention by providing a computer-implemented method for determining a BODE index value for a patient. The method includes using a Body Mass Index measuring device to measure Body Mass Index of the patient to obtain Body Mass Index data; using an airway obstruction measuring device to measure airway obstruction of the patient to obtain airway obstruction data; using a respiration rate sensor to measure a respiration rate of the patient and to obtain respiration rate data; using an activity monitor to measure physical activity of the patient and to obtain physical activity data; and executing, on one or more computer processors, one or more computer program modules to determine the BODE index value for the patient based on the Body Mass Index data, the airway obstruction data, the respiration rate data, and the physical activity data.

Another aspect of the present invention provides a system for determining a BODE index value for a patient. The system includes a Body Mass Index measuring device, an airway obstruction measuring device, at least one sensor, and at least one processor. The Body Mass Index measuring device is configured to measure Body Mass Index of the patient to obtain Body Mass Index data. The airway obstruction measuring device is configured to measure airway obstruction of the patient to obtain airway obstruction data. The sensor is configured to measure a) a respiration rate of the patient to obtain respiration rate data, and b) physical activity of the patient to obtain physical activity data. The processor is configured to process the Body Mass Index data, the airway obstruction data, the respiration rate data, and the physical activity data to determine the BODE index value of the patient.

Another aspect of the present invention provides an apparatus for determining a BODE index value for a patient. The apparatus includes a Body Mass Index measuring means for measuring Body Mass Index of the patient to obtain Body Mass Index data; an airway obstruction measuring means for measuring airway obstruction of the patient to obtain airway obstruction data; at least one sensing means for measuring a) a respiration rate of the patient to obtain respiration rate data; and b) physical activity of the patient to obtain physical activity data; and a means for processing the Body Mass Index data, the airway obstruction data, the respiration rate data, and the physical activity data to determine the BODE index value of the patient.

These and other aspects of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. It shall also be appreciated that the features of one embodiment disclosed herein can be used in other embodiments disclosed herein. As used in the specification and in the claims, the singular form of “a”, “an”, and “the” include plural referents unless the context clearly dictates otherwise.

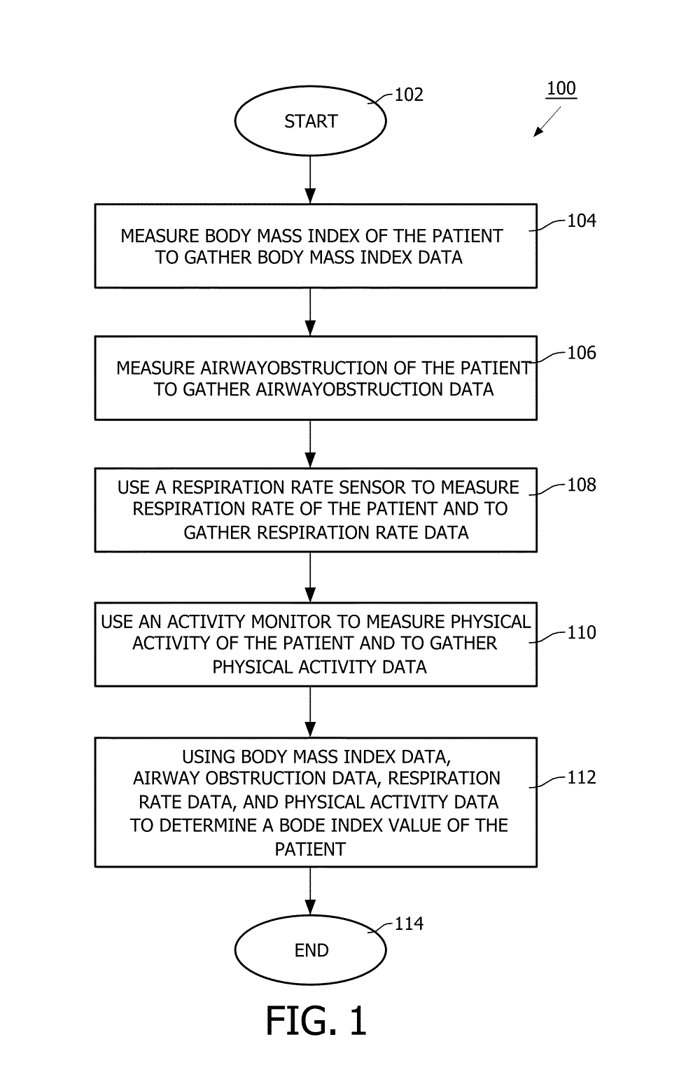

FIG. 1 is a flow chart illustrating a method for determining a BODE index value for a patient in accordance with an embodiment of the present invention;

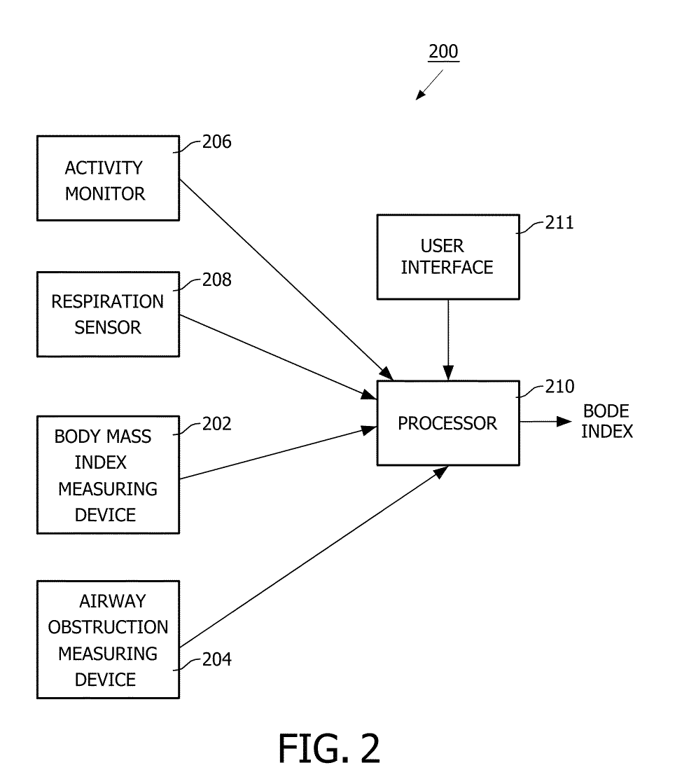

FIG. 2 shows a system for determining the BODE index value for the patient in accordance with an embodiment of the present invention;

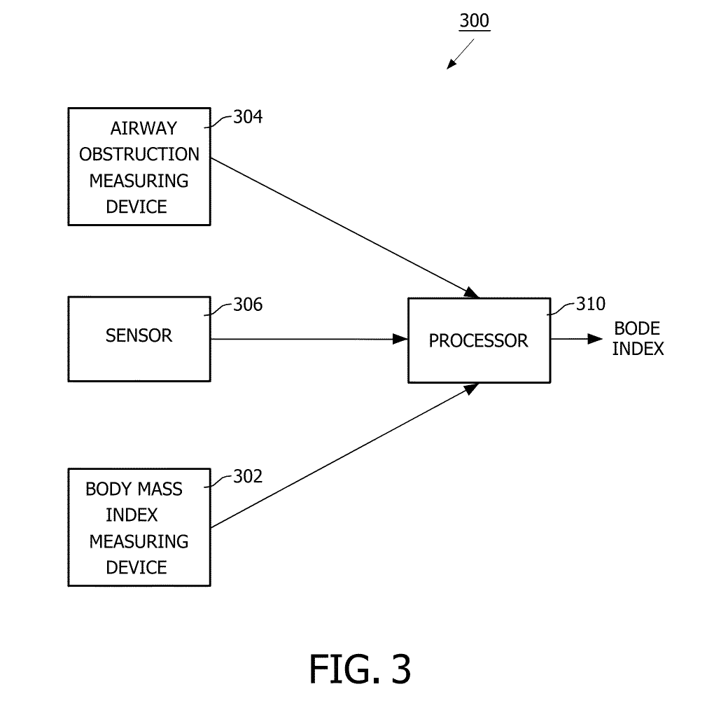

FIG. 3 shows a system determining the BODE index value for the patient in accordance with another embodiment of the present invention; and

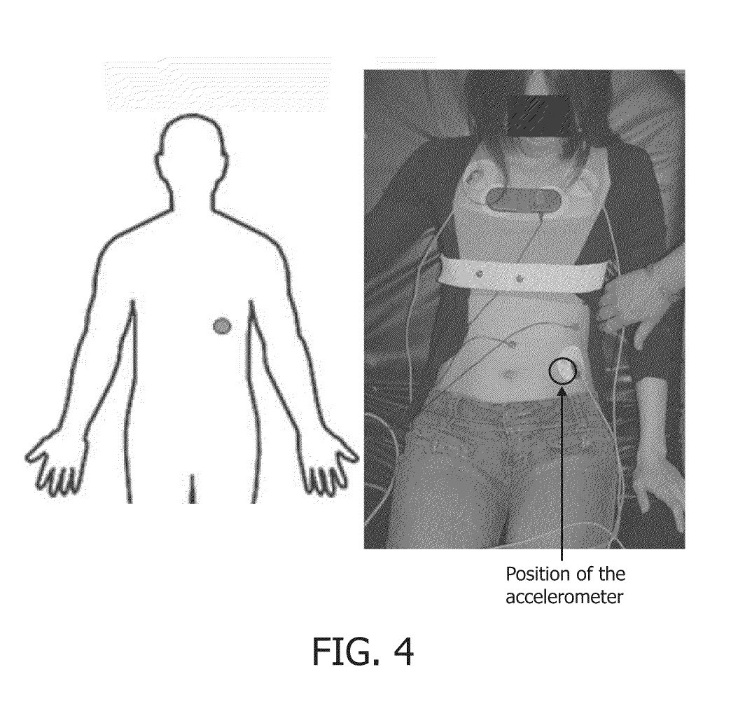

FIG. 4 shows the positioning of a sensor (e.g., an accelerometer) in accordance with an embodiment of the present invention.

FIG. 1 is a flow chart illustrating a computer-implemented method for determining a BODE index value for a in accordance with an embodiment of the present invention. The method 100 enables continuous measurement of the BODE Index, for example, in the home environment of the patient. The method 100 uses an integrated set of sensors to measure four parameters (i.e., the Body Mass Index (B), degree of obstruction (O), dyspnea (D), and exercise capacity (E)) that correlate with the BODE Index. The method 100 is implemented in a computer system comprising one or more processors 210 (as shown in and explained with respect to FIG. 2) configured to execute one or more computer programs modules. In one embodiment, the processor 210 (as shown in and explained with respect to FIG. 2) can comprise either one or a plurality of processors therein.

The method 100 begins at procedure 102. At procedure 104, a Body Mass Index of the patient is measured to obtain Body Mass Index data. In one embodiment, the Body Mass Index of the patient is measured using a Body Mass Index measuring device 202 (as shown in and described with reference to FIG. 2), or 302 (as shown in and described with reference to FIG. 3).

At procedure 106, an airway obstruction of the patient is measured to obtain airway obstruction data. In one embodiment, the airway obstruction of the patient is measured using an airway obstruction measuring device 204 (as shown in and described with reference to FIG. 2), or 304 (as shown in and described with reference to FIG. 3).

At procedure 108, a respiration rate sensor 206 (as shown in and described with reference to FIG. 2), or 306 (as shown in and described with reference to FIG. 3) is used to measure the respiration rate of the patient and to obtain the respiration rate data. At procedure 110, an activity monitor 208 (as shown in and described with reference to FIG. 2), or 308 (as shown in and described with reference to FIG. 3) is used to measure physical activity of the patient and to obtain the physical activity data.

In one embodiment, the physical activity, and the respiration rate of the patient may be measured using separate sensors as described with respect to a system 200 (as shown in FIG. 2). In another embodiment, a single sensor, such as the sensor 306 (as shown in and described with reference to FIG. 3) may be used to measure both the physical activity, and the respiration rate of the patient.

At procedure 112, a processor 210 (as shown in and described with reference to FIG. 2), or 310 (as shown in and described with reference to FIG. 3) is configured to use the Body Mass Index data, the airway obstruction data, the respiration rate data, and the physical activity data to determine the BODE index value of the patient. In other words, the data from the various sensors are subsequently converted to the BODE Index value to provide an objective and continuous measure of the BODE index. The method 100 ends at procedure 114. In one embodiment, the procedures 102-114 can be performed by one or more computer program modules that can be executed by one or more processors 210 (as shown in and explained with respect to FIG. 2).

FIG. 2 shows system 200 for determining the BODE index value for the patient in accordance with one embodiment. In one embodiment, system 200 may be used by the patient in his/her home environment. System 200 may include the Body Mass Index measuring device 202, airway obstruction measuring device 204, activity monitor 206, respiration sensor 208, and processor 210. In one embodiment, processor 210 can comprise either one or a plurality of processors therein. In one embodiment, processor 210 can be a part of or forming a computer system.

In one embodiment, system 200 may include a user interface 211, which is in communication with processor 210. User interface 211 is configured to accept input from the patient (or caregiver), and to transmit (and display) output of system 200. In one embodiment, user interface 211 may include a keypad that allows the patient or caregiver to input information (e.g., patient's weight, patient's BMI, FEV₁ value, or peak flow meter readings) into processor 210 to determine the BODE index value of the patient. In one embodiment, user interface 211 may include a display screen that provides a visual data output (e.g., the BODE index value) to the patient.

In one embodiment, user interface 211 may be a graphical user interface. In one embodiment, user interface 211 may be provided integral with the sensor set (i.e., activity monitor and/or the respiration rate sensor). In another embodiment, the user interface 211 may be provided remote from or proximal to the sensor set.

Body Mass Index (or BMI) is generally calculated using a mathematical formula that takes into account patient's body weight and height. BMI is obtained by dividing patient's body weight by the square of patient's height. For example, when measuring the BMI in metric system, the patient's weight is measured in kilograms, while the height of the patient is measured in meters.

In one embodiment, the Body Mass Index measuring device may include a weighing or a weight scale. The patient's weight measured using the weight scale may be input into processor 210 using user interface 211. The patient's height is also measured and may be input into processor 210 using user interface 211. In one embodiment, the patient's height is measured using a height measuring scale. In one embodiment, the height measuring scale and the weight scale may be integral to form a single device. In one embodiment, the weight scale and/or the height measuring scale is electronic and electronically inputs the data (i.e., patient's weight and/or the patient's height) into processor 210 without the need for the patient to use user interface 211. Processor 210 is configured to calculate the Body Mass Index, for example, using the mathematical formula (discussed above) that is saved in the processor or an associated memory (not shown).

In another embodiment, the Body Mass Index measuring device 202 may include a BMI scale. For example, in one embodiment, the BMI scale may be a Taylor 5700 Body Mass Index (BMI) scale available from Taylor, or Seca 882 Remote Display BMI scale available from Seca. In such an embodiment, the patient may input the BMI value obtained from the BMI scale into processor 210 using user interface 211. Or, as noted above, the data may be automatically and directly input into processor 210 without the need for input via user interface 211.

The BMI scale and the weight scale described above are just two examples for measuring the Body Mass Index data, however, it is contemplated that other Body Mass Index measuring devices known in the art may be used to measure the Body Mass Index data of the patient.

As noted above, the Body Mass Index measuring device 202 may be directly connected to processor 210. In such an embodiment, the Body Mass Index measuring device 202 may be connected to the processor over a wired or wireless network, for example.

In one embodiment, the airway obstruction is measured using airway obstruction measuring device 204. In one embodiment, the airway obstruction measuring device 204 may include a spirometer. In one embodiment, the spirometer is a handheld spirometer. For example, the handheld spirometer may be a MicroGP spirometer available from Micro Direct, Inc. The spirometer may include an analog spirometer or a digital spirometer.

In one embodiment, the airway obstruction is assessed using a spirometry test. During the spirometry test, patient breathes into a mouth piece that is connected to a spirometer. The spirometer is configured to record the amount and the rate of air that patient breathes in and out over a period of time. In one embodiment, during the spirometry test, the patient takes the deepest breath they can and exhales as hard as possible for as long as they are able to. In one embodiment, the spirometry test is normally repeated three times to ensure reproducibility.

The spirometer measures Forced Expiratory Volume measured over one second (FEV₁) (i.e., volume expired in the first second of maximal expiration after a maximal inspiration). FEV₁ is a measure of how quickly the lungs can be emptied. A lower FEV₁ value generally indicates a greater degree of airway obstruction.

The spirometer is also configured to measure Forced Vital Capacity (FVC) (i.e., maximum volume of air that can be exhaled during a forced maneuver), and Peak Expiratory Flow (PEF). The spirometer is also configured to measure FEV₁/FVC (i.e., FEV₁ expressed as a percentage of the FVC). FEV₁/FVC provides a clinically useful index of airflow limitation. For example, a value of FEV₁/FVC less than 70% indicates airflow limitation and the possibility of COPD.

The airway obstruction grading is illustrated in the TABLE. 2. TABLE. 2 shows the FEV₁ as a percentage of a predicted value. The predicted value is determined based on patient's age, patient's sex, patient's height, patient's weight, and/or patient's race.

TABLE 2

| | | | Very severe |
|---|---|---|---|
| Mild airway | Moderate airway | Severe airway | airway |
| obstruction | obstruction | obstruction | obstruction |
| FEV₁ ≧ 80% | 50% of a | 30% of a predicted | FEV₁ < 30% |
| of a | predicted | value ≦ FEV₁ < | of a |
| predicted | value ≦ FEV₁ < | 50% of a predicted | predicted |
| value | 80% of a | value | value |
| | predicted | | |
| | value | | |

If the measured FEV₁ value is greater than 80% of the predicted value, then the patient may have a mild airway obstruction. If the measured FEV₁ value is between 50% and 80%, then the patient may have a moderate airway obstruction. If the measured FEV₁ value is between 30% and 50%, then the patient may have a severe airway obstruction. If the measured FEV₁ value is less than 30%, then the patient may have a very severe airway obstruction.

In another embodiment, airway obstruction device 204 may include a peak flow meter. The peak flow meter is configured to measure the patient's maximum speed of expiration, or peak expiratory flow (PEFR or PEF). The peak flow readings are higher when patient's airways are not constricted, and lower when the patient's airways are constricted. The spirometer and the peak flow meter described above are just two examples for measuring the airway obstruction data, however, it is contemplated that other airway obstruction measuring devices known in the art may be used to measure the airway obstruction data of the patient.

In one embodiment, airway obstruction device 204 may be directly connected to the processor 210. In such an embodiment, the airway obstruction device 204 may be connected to the processor 210 over a wired or wireless network, for example.

In another embodiment, the patient may manually input the airway obstruction data (i.e., the FEV₁ value obtained from the spirometer, or the peak flow readings obtained from the peak flow meter) obtained from airway obstruction device 204 into processor 210 using user interface 211.

Activity monitor 206 is configured to detect body movements of the patient such that a signal from the activity monitor is correlated to the level of a patient's physical activity. In one embodiment, the activity monitor may include an accelerometer, such as a three-axis accelerometer. Such an accelerometer may include a sensing element that is configured to determine acceleration data in at least three axes. For example, in one embodiment, the three-axis accelerometer may be a three-axis accelerometer (i.e., manufacturer part number: LIS3L02AQ) available from STMicroelectronics.

In another embodiment, activity monitor 206 may be a piezoelectric sensor. The piezoelectric sensor may include a piezoelectric element that is sensitive to body movements of the patients. In one embodiment, activity monitor 206 may be positioned, for example, at the thorax of the patient or at the abdomen of the patient.

The accelerometer and the piezoelectric sensor described above are just two examples for measuring the physical activity data, however, it is contemplated that other activity monitors known in the art may be used to measure the physical activity data of the patient.

In one embodiment, respiration rate sensor 208, which is configured to measure the respiration pattern of the patient, may include an accelerometer or a microphone. In one embodiment, the accelerometer may be a three-axis accelerometer. For example, the three-axis accelerometer may be a three-axis accelerometer (i.e., manufacturer part number: LIS3L02AQ) available from STMicroelectronics.

In another embodiment, a microphone is constructed and arranged to receive sound of inspiration of the patient in order to determine the respiration rate of the patient. In one embodiment, respiration rate sensor 208 may be a Respiband™ available from Ambulatory Monitoring, Inc. of Ardsley, N.Y.

In yet another embodiment, respiration rate sensor 208 may include a chest band and a microphone as described in U.S. Pat. No. 6,159,147, hereby incorporated by reference. In such an embodiment, the chest band may be placed around a patient's chest to measure the patient's respiration rate, for example.

The accelerometer, the microphone, and the combination of the chest band and the microphone described above are just few examples for measuring the respiration rate data, however, it is contemplated that other respiration rate sensors known in the art may be used to measure the respiration rate data of the patient.

In one embodiment, activity monitor 206 and/or respiration rate sensor 208 may be directly connected to processor 210. In other words, the data from activity monitor 206 and/or respiration rate sensor 208 may be automatically and directly input into processor 210 without the need for input via user interface 211. In such an embodiment, the activity monitor and/or the respiration rate sensor may be connected to the processor 210 over a wired or wireless network, for example.

Processor 210 is configured to process the Body Mass Index data, the airway obstruction data, the respiration rate data, and the physical activity data to determine the BODE index value of the patient. In one embodiment, system 200 may include a single processor to process the Body Mass Index data, the airway obstruction data, the respiration rate data, and the physical activity data to determine the BODE index value of the patient.

In another embodiment, system 200 may include multiple processors, where each processor is configured to perform a specific function or operation. In such an embodiment, the multiple processors may be configured to process the Body Mass Index data, the airway obstruction data, the respiration rate data, and the physical activity data to determine the BODE index value of the patient.

In one embodiment, processor 210 is configured to receive input information (e.g., patient's body weight and height) from the patient and to further process this input information to obtain the Body Mass Index data. In other words, as explained above, processor 210 is configured to calculate the Body Mass Index, for example, using the mathematical formula (discussed above) that is saved in the processor. In another embodiment, processor 210 is configured to receive the Body Mass Index data from Body Mass Index measuring device 202.

Processor 210 is configured to receive the airway obstruction data from airway obstruction measuring device 204, the physical activity data from activity monitor 206, and the respiration rate data from respiration rate sensor 208.

A point value is assigned to each parameter (i.e., Body Mass Index data (B), airway obstruction data (or degree of obstruction) (O), respiration rate data (or dyspnea) (D), and the physical activity data (or exercise capacity) (E)) and these point values are stored in the processor 210. Processor 210 is configured to add the point values obtained for each parameter (i.e., Body Mass Index (B), degree of obstruction (O), dyspnea (D), and exercise capacity (E)) to obtain the BODE index of the patient. The BODE index generally ranges from 0-10 points, with higher scores indicating a greater risk of death, see TABLE. 3 below.

TABLE 3

| | Points | | | | |
|---|---|---|---|---|---|
| BODE parameters | 1 | 2 | 3 | 4 | Score |
| Body Mass Index data | <21 | ≧21 | | | |
| measured using the | | | | | |
| Body Mass Index | | | | | |
| measuring device | | | | | |
| Airway obstruction | >65% | 54-64% | 36-49% | ≦35% | |
| data (measured | | | | | |
| in FEV₁) | | | | | |
| measured using the | | | | | |
| airway obstruction | | | | | |
| measuring device | | | | | |
| Respiration rate | 14-17 | 18-21 | 22-25 | >25 | |
| data measured using | breaths/ | breaths/ | breaths/ | breaths/ | |
| the respiration rate | minute | minute | minute | minute | |
| sensor Physical | ≧350 m | 250-349 m | 150-249 m | ≦149 m | |
| activity data | | | | | |
| (distance | | | | | |
| measured in | | | | | |
| meters) measured | | | | | |
| using the activity | | | | | |
| monitor | | | | | |
| | | | | Total | |

In one embodiment, the respiration rate data measured using the respiration rate sensor may be correlated to each dyspnea scale (i.e., normally used in the BODE scoring card, see TABLE. 1).

In one embodiment, the activity monitor replaces the six minute walk distance test by correlating the free living activity with a certain predicted distance that the patient can walk in six minutes.

FIG. 3 shows a system 300 for determining a BODE index value for the patient in accordance with another embodiment of the present invention. System 300 includes a Body Mass Index measuring device 302, an airway obstruction measuring device 304, a sensor 306, and a processor 310. System 300 is similar to system 200 described with respect to FIG. 2, except for the following aspects.

Instead of using separate sensors to measure the physical activity, and the respiration rate of the patient (e.g., as described with respect to system 200 in FIG. 2), system/method 300 uses a single sensor 306 to measure both the physical activity, and the respiration rate of the patient.

In one embodiment, sensor 306 may be positioned, for example, at the thorax of the patient or at the abdomen of the patient. In one embodiment, as shown in FIG. 4, the accelerometer is positioned at the lower ribs, roughly halfway between the central and the lateral position. The positioning of the accelerometer is shown in FIG. 4 allows reliable monitoring of both the respiration rate as well as the physical activity. In another embodiment, sensor 306 may be positioned such that the sensor 306 is in close proximity with at least a portion of the patient's body.

In one embodiment, sensor 306 may be a part of a wearable band (that may be worn on any portion of the patient's body) or may be part of a wearable garment worn by the patient. In one embodiment, sensor 306 may be directly connected to processor 310. In other words, the data from the sensor may be automatically and directly input into the processor without the need for input via a user interface. In such an embodiment, sensor 306 may be connected to processor 310 over a wired or wireless network, for example.

In one embodiment, processor 310 is configured to 1) receive acceleration data in at least three axes from sensor or accelerometer 306; 2) determine the respiration rate data from the accelerometer data; 3) determine physical activity data associated with the respiration rate data; and 4) analyze the physical activity data, and the respiration data (i.e., along with the body mass index data and the airway obstruction data) to determined a BODE index value for the patient.

In one embodiment, system 300 may include a single processor to process the Body Mass Index data, the airway obstruction data, the respiration rate data, and the physical activity data to determine the BODE index value of the patient. In another embodiment, system 300 may include multiple processors, where each processor is configured to perform a specific function or operation. In such an embodiment, the multiple processors may be configured to process the Body Mass Index data, the airway obstruction data, the respiration rate data, and the physical activity data to determine the BODE index value of the patient.

In one embodiment, system 200 or 300 may also be configured to transmit the BODE index value to the health care provider over a network (wired or wireless, for example).

Another aspect of the present invention provides an apparatus for determining a BODE index value for a patient. The apparatus includes a Body Mass Index measuring means for measuring Body Mass Index of the patient to obtain Body Mass Index data; an airway obstruction measuring means for measuring airway obstruction of the patient to obtain airway obstruction data; at least one sensing means for measuring a) a respiration rate of the patient to obtain respiration rate data; and b) physical activity of the patient to obtain physical activity data; and a means for processing the Body Mass Index data, the airway obstruction data, the respiration rate data, and the physical activity data to determine the BODE index value of the patient.

Embodiments of the invention, such as the processors, for example, may be made in hardware, firmware, software, or various combinations thereof. The invention may also be implemented as instructions stored on a machine-readable medium, which may be read and executed using one or more processing devices. In one embodiment, the machine-readable medium may include various mechanisms for storing and/or transmitting information in a form that may be read by a machine (e.g., a computing device). For example, a machine-readable storage medium may include read only memory, random access memory, magnetic disk storage media, optical storage media, flash memory devices, and other media for storing information, and a machine-readable transmission media may include forms of propagated signals, including carrier waves, infrared signals, digital signals, and other media for transmitting information. While firmware, software, routines, or instructions may be described in the above disclosure in terms of specific exemplary aspects and embodiments performing certain actions, it will be apparent that such descriptions are merely for the sake of convenience and that such actions in fact result from computing devices, processing devices, processors, controllers, or other devices or machines executing the firmware, software, routines, or instructions.

Although the invention has been described in detail for the purpose of illustration, it is to be understood that such detail is solely for that purpose and that the invention is not limited to the disclosed embodiments, but, on the contrary, is intended to cover modifications and equivalent arrangements that are within the spirit and scope of the appended claims. In addition, it is to be understood that the present invention contemplates that, to the extent possible, one or more features of any embodiment can be combined with one or more features of any other embodiment.

1. A computer-implemented method for determining a BODE index value for a patient, the method comprising:
using a Body Mass Index measuring device to measure Body Mass Index of the patient to obtain Body Mass Index data;
using an airway obstruction measuring device to measure airway obstruction of the patient to obtain airway obstruction data;
using a respiration rate sensor to measure a respiration rate of the patient and to obtain respiration rate data;
correlating the respiration rate data to a score on a dyspnea scale;
using an activity monitor to measure physical activity of the patient and to obtain physical activity data; and
executing, on one or more computer processors, one or more computer program modules to determine the BODE index value for the patient based on the Body Mass Index data, the airway obstruction data, the score on the dyspnea scale that is correlated to the respiration rate data, and the physical activity data.

2. The method of claim 1, wherein respiration rates between 14 and 17 breaths per minute correlate to a score of 0 on the dyspnea scale, wherein respiration rates between 18 and 21 breaths per minute correlate to a score of 1 on the dyspnea scale, wherein respiration rates between 22 and 25 breaths per minute correlate to a score of 2 on the dyspnea scale, and wherein respiration rates over 25 breaths per minute correlate to a score of 3 on the dyspnea scale.

3. The method of claim 1, wherein the respiration rate data from the respiration rate sensor is automatically input into the one or more computer program modules to determine the BODE index value for the patient.

4. The method of claim 1, wherein the airway obstruction data from the airway obstruction measuring device is automatically input into the one or more computer program modules to determine the BODE index value for the patient.

5. The method of claim 1, wherein determinations of the BODE index value for the patient are performed continuously in a home environment of the patient.

6. The method of claim 1, wherein the activity monitor comprises an accelerometer, wherein the respiration rate sensor comprises the accelerometer, and wherein the accelerometer is part of a wearable band or a wearable garment.

7. A system for determining a BODE index value for a patient, the system comprising:
(a) a Body Mass Index measuring device configured to measure Body Mass Index of the patient to obtain Body Mass Index data;
(b) an airway obstruction measuring device configured to measure airway obstruction of the patient to obtain airway obstruction data;
(c) a sensor configured to measure:
1) a respiration rate of the patient to obtain respiration rate data,
2) physical activity of the patient to obtain physical activity data, or
3) both respiration rate and physical activity; and
(d) at least one processor configured to
1) process the respiration rate data to determine a score on a dyspnea scale that correlates to the respiration rate data; and
2) process the Body Mass Index data, the airway obstruction data, the score on the dyspnea scale that is correlated to the respiration rate data, and the physical activity data to determine the BODE index value for the patient.

8. The system of claim 7, wherein respiration rates between 14 and 17 breaths per minute correlate to a score of 0 on the dyspnea scale, wherein respiration rates between 18 and 21 breaths per minute correlate to a score of 1 on the dyspnea scale, wherein respiration rates between 22 and 25 breaths per minute correlate to a score of 2 on the dyspnea scale, and wherein respiration rates over 25 breaths per minute correlate to a score of 3 on the dyspnea scale.

9. The system of claim 7, wherein the respiration rate data from the sensor is automatically input into the at least one processor to determine the BODE index value for the patient.

10. The system of claim 7, wherein the airway obstruction data from the airway obstruction measuring device is automatically input into the at least one processor to determine the BODE index value for the patient.

11. The system of claim 7, wherein determinations of the BODE index value for the patient are performed continuously in a home environment of the patient.

12. The system of claim 7, wherein the sensor comprises an accelerometer that measures both the respiration rate of the patient and the physical activity of the patient, and wherein the accelerometer is part of a wearable band or a wearable garment.

13. An apparatus for determining a BODE index value for a patient, the apparatus comprising:
(a) Body Mass Index measuring means for measuring Body Mass Index of the patient to obtain Body Mass Index data;
(b) airway obstruction measuring means for measuring airway obstruction of the patient to obtain airway obstruction data;
(c) sensing means for measuring:
1) a respiration rate of the patient to obtain respiration rate data,
2) physical activity of the patient to obtain physical activity data, or
3) both the respiration rate and the physical activity; and
(d) means for processing
1) the respiration rate data to determine a score on a dyspnea scale that correlates to the respiration rate data; and
2) the Body Mass Index data, the airway obstruction data, the score on the dyspnea scale that is correlated to the respiration rate data, and the physical activity data to determine the BODE index value for the patient.

14. The apparatus of claim 13, wherein respiration rates between 14 and 17 breaths per minute correlate to a score of 0 on the dyspnea scale, wherein respiration rates between 18 and 21 breaths per minute correlate to a score of 1 on the dyspnea scale, wherein respiration rates between 22 and 25 breaths per minute correlate to a score of 2 on the dyspnea scale, and wherein respiration rates over 25 breaths per minute correlate to a score of 3 on the dyspnea scale.

15. The apparatus of claim 13, wherein the respiration rate data from the sensing means is automatically input into the means for processing to determine the BODE index value for the patient.

16. The apparatus of claim 13, wherein the airway obstruction data from the airway obstruction measuring means is automatically input into the means for processing to determine the BODE index value for the patient.

17. The apparatus of claim 13, wherein determinations of the BODE index value for the patient are performed continuously in a home environment of the patient.

18. The apparatus of claim 13, wherein the sensing means comprises an accelerometer that measures both the respiration rate of the patient and the physical activity of the patient, and wherein the accelerometer is part of a wearable band or a wearable garment.
